(19) United States
(12) Patent Application Publication
(10) Pub. No.: US 2022/0310272 A1
(43) Pub. Date: Sep. 29, 2022

(54) APPLICATION FOR ANONYMOUS SHARING OF ALLERGY AND/OR HEALTH INFORMATION

(71) Applicant: Calmerge, LLC, Edmond, OK (US)

(72) Inventor: Deana M. Glenn, Oklahoma City, OK (US)

(21) Appl. No.: 17/703,391

(22) Filed: Mar. 24, 2022

Related U.S. Application Data
(60) Provisional application No. 63/165,353, filed on Mar. 24, 2021.

(51) Int. Cl. G16H 70/20

(52) U.S. Cl. CPC G16H 70/20

(57) ABSTRACT

A method of establishing communication between a host system and a plurality of user devices each having an input device; accepting, via the input device of a first one of the plurality of user devices associated with a first user, input comprising user information, allergy information including an allergy and a severity of the allergy, and at least one group associated with the first user; storing, the user information, allergy information, and the at least one group in a user profile of the first user; automatically generating an alert, the alert containing information indicative of the allergy and the severity of the allergy but none of the user information; and automatically transmitting the alert to at least one second one of the plurality of user devices associated with at least one second member of the at least one group associated with the first user.

### CROSS-REFERENCE TO RELATED APPLICATIONS

The present patent application hereby incorporates by reference the entire provisional patent application identified by U.S. Ser. No. 63/165,353, filed on Mar. 24, 2021, titled “APPLICATION FOR ANONYMOUS SHARING OF ALLERGY AND/OR HEALTH INFORMATION” and claims priority thereto under 35 U.S.C. § 119(e).

### BACKGROUND

Allergies are our body's immune response to a normally harmless substance such as pollen, molds, animal dander, latex, certain foods, insect stings, medicines, and chemicals, for instance. Allergy symptoms range from mild (rash, itching, rhinitis), to severe (abdominal pain, nausea, vomiting, diarrhea, chest pain/tightness, trouble swallowing/breathing, swelling of face/eyes/tongue, facial flushing, dizziness, wheezing, heart palpitations), to life-threatening anaphylaxis. Treatments include antihistamines, decongestants, nasal steroids, asthma medicines, and immunotherapy.

Food allergies present a real danger and members of a group may not be aware of another member's allergy or a severity of a reaction to that allergy. Although those close to a person who has allergies are likely aware of the allergies and associated reactions, there are situations when allergy information may be important but may not have been properly communicated. For instance, when a child participates in sports, it is typical for one or more parents to bring snacks and/or drinks to a team event to share. This may lead to an inadvertent allergic reaction when one of the children eats a snack containing an ingredient to which they are allergic.

Because a reaction to even a small amount of an allergen can be potentially fatal, those with allergies and their caregivers must take great care to avoid allergens, watch for symptoms, and keep track of treatments (e.g., epinephrine pens, antihistamines, decongestants, nasal steroids, asthma medicines, etc.). However, there are times when the person with the allergy is in the care of others such as when they are at school, participating in sporting activities, at work, at church, gatherings with family and/or friends, etc. In such cases, it would be beneficial for allergy information to be available to other members of the group and group leaders to help prevent exposure to known allergens, allergic reactions, doctor's visits, hospitalizations, and potentially death. However, the information needs to be shared in such a way that the person with the allergy is not excluded or stigmatized, either intentionally or unintentionally.

Therefore, a need exists for a system and method of anonymously sharing allergy information, which may include severity of reaction and/or possible treatments, with a group. It is to such an improved system and method that the presently disclosed inventive concepts are directed.

### BRIEF DESCRIPTION OF THE SEVERAL VIEWS OF THE DRAWINGS

To assist those of ordinary skill in the relevant art in making and using the subject matter hereof, reference is made to the appended drawings, which are not intended to be drawn to scale, and in which like reference numerals are intended to refer to similar elements for consistency. For purposes of clarity, not every component may be labeled in every drawing.

FIG. 1 is a diagrammatic view of hardware forming an exemplary embodiment of a system for anonymously sharing allergy information constructed in accordance with the present disclosure.

FIG. 2 is a diagrammatic view of an exemplary user device for use in the system for anonymously sharing allergy information illustrated in FIG. 1.

FIG. 3 is a diagrammatic view of an exemplary embodiment of a host system for use in the system for anonymously sharing allergy information illustrated in FIG. 1.

FIG. 4 is an illustration of an exemplary get started screen in accordance with some embodiments of the present disclosure.

FIG. 5 is an illustration of an exemplary information input screen in accordance with some embodiments of the present disclosure.

FIG. 6 is an illustration of an exemplary group allergy list screen in accordance with some embodiments of the present disclosure.

FIG. 7 is an illustration of an exemplary allergy severity screen in accordance with some embodiments of the present disclosure.

FIG. 7A is an illustration of an exemplary mild symptom screen in accordance with some embodiments of the present disclosure.

FIG. 7B is an illustration of an exemplary severe symptom screen in accordance with some embodiments of the present disclosure.

FIG. 7C is an illustration of an exemplary epinephrine pen pop-up screen of the severe symptom screen of FIG. 7B in accordance with some embodiments of the present disclosure.

FIG. 7D is an illustration of an exemplary anaphylaxis screen in accordance with some embodiments of the present disclosure.

FIG. 8 is an illustration of an exemplary allergy severity input screen in accordance with some embodiments of the present disclosure.

FIG. 9 is an illustration of an exemplary group members screen in accordance with some embodiments of the present disclosure.

FIG. 10 is an illustration of an exemplary menu screen in accordance with some embodiments of the present disclosure.

FIG. 11 is an illustration of an exemplary user profile screen in accordance with some embodiments of the present disclosure.

FIG. 12 is an illustration of an exemplary add reaction screen in accordance with one embodiment of the present disclosure.

FIG. 13 is an illustration of an exemplary snack suggestion screen in accordance with one embodiment of the present disclosure.

FIG. 14 is an illustration of an exemplary add snack suggestion screen in accordance with one embodiment of the present disclosure.

FIG. 15 is an illustration of an exemplary snack schedule screen in accordance with one embodiment of the present disclosure.

FIG. 16 is an illustration of an exemplary assign snack date screen in accordance with one embodiment of the present disclosure.

FIG. 17 is an illustration of an exemplary snack schedule screen in accordance with one embodiment of the present disclosure.

FIG. 18 is an illustration of an exemplary child information screen in accordance with one embodiment of the present disclosure.

FIG. 19 is an illustration of an exemplary health status screen in accordance with one embodiment of the present disclosure.

FIG. 20 is an illustration of a notification screen in accordance with one embodiment of the present disclosure.

FIG. 21 is an illustration of a health status screen in accordance with one embodiment of the present disclosure.

FIG. 22 is an illustration of a calendar screen in accordance with one embodiment of the present disclosure.

FIG. 23 is an illustration of a student list screen in accordance with one embodiment of the present disclosure.

### DETAILED DESCRIPTION

Before explaining at least one embodiment of the disclosure in detail, it is to be understood that the disclosure is not limited in its application to the details of construction, experiments, exemplary data, and/or the arrangement of the components set forth in the following description or illustrated in the drawings unless otherwise noted.

The systems and methods as described in the present disclosure are capable of other embodiments or of being practiced or carried out in various ways. Also, it is to be understood that the phraseology and terminology employed herein is for purposes of description, and should not be regarded as limiting.

The following detailed description refers to the accompanying drawings. The same reference numbers in different drawings may identify the same or similar elements.

As used in the description herein, the terms “comprises,” “comprising,” “includes,” “including,” “has,” “having,” or any other variations thereof, are intended to cover a non-exclusive inclusion. For example, unless otherwise noted, a process, method, article, or apparatus that comprises a list of elements is not necessarily limited to only those elements, but may also include other elements not expressly listed or inherent to such process, method, article, or apparatus.

Further, unless expressly stated to the contrary, “or” refers to an inclusive and not to an exclusive “or”. For example, a condition A or B is satisfied by one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concept. This description should be read to include one or more, and the singular also includes the plural unless it is obvious that it is meant otherwise. Further, use of the term “plurality” is meant to convey “more than one” unless expressly stated to the contrary.

As used herein, any reference to “one embodiment,” “an embodiment,” “some embodiments,” “one example,” “for example,” or “an example” means that a particular element, feature, structure or characteristic described in connection with the embodiment is included in at least one embodiment. The appearance of the phrase “in some embodiments” or “one example” in various places in the specification is not necessarily all referring to the same embodiment, for example.

Circuitry, as used herein, may be analog and/or digital components, or one or more suitably programmed processors (e.g., microprocessors) and associated hardware and software, or hardwired logic. Also, “components” may perform one or more functions. The term “component” may include hardware, such as a processor (e.g., microprocessor), a combination of hardware and software, and/or the like. Software may include one or more computer executable instructions that when executed by one or more components cause the component to perform a specified function. It should be understood that the algorithms described herein may be stored on one or more non-transitory memory. Exemplary non-transitory memory may include random access memory, read only memory, flash memory, and/or the like. Such non-transitory memory may be electrically based, optically based, and/or the like.

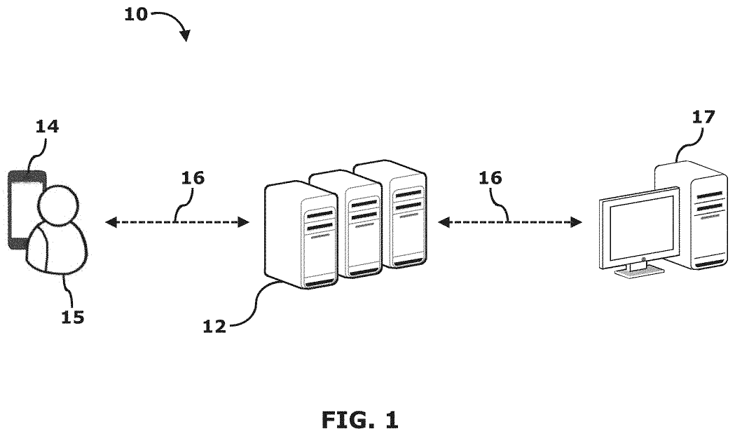

Referring now to the Figures, and in particular to FIG. 1, shown therein is a diagrammatic view of hardware forming an exemplary embodiment of a system 10 for anonymously sharing allergy information constructed in accordance with the present disclosure.

The system 10 may be provided with at least one host system 12 (hereinafter “host system 12”), a plurality of user devices 14 (hereinafter “user device 14”), and a network 16. In some embodiments, the system 10 may include at least one external system 17 (hereinafter “external system 17”) for use by an administrator to add, delete, or modify user information, provide management reporting, or manage user information. The system 10 may be a system or systems that are able to embody and/or execute the logic of the processes described herein. Logic embodied in the form of software instructions and/or firmware may be executed on any appropriate hardware. For example, logic embodied in the form of software instructions and/or firmware may be executed on a dedicated system or systems, on a personal computer system, on a distributed processing computer system, and/or the like. In some embodiments, logic may be implemented in a stand-alone environment operating on a single computer system and/or logic may be implemented in a networked environment such as a distributed system using multiple computers and/or processors as depicted in FIG. 1, for example.

The host system 12 of the system 10 may include a single processor or multiple processors working together or independently to perform a task. In some embodiments, the host system 12 may be partially or completely network-based or cloud based. The host system 12 may or may not be located in single physical location. Additionally, multiple host systems 12 may or may not necessarily be located in a single physical location.

In some embodiments, the system 10 may be distributed, and include at least one host system 12 communicating with one or more user device 14 via the network 16. As used herein, the terms “network—based,” “cloud-based,” and any variations thereof, are intended to include the provision of configurable computational resources on demand via interfacing with a computer and/or computer network, with software and/or data at least partially located on a computer and/or computer network.

In some embodiments, the network 16 may be the Internet and/or other network. For example, but not by way of limitation, if the network 16 is the Internet, a primary user interface of the system 10 may be delivered through a series of web pages or private internal web pages of a company or corporation, which may be written in hypertext markup language. One having ordinary skill in the art would understand that the primary user interface of the system 10 may be, but is not limited to being, another type of interface including, but not limited to, a Windows-based application, a tablet-based application, a mobile web interface, an application running on a mobile device, and/or the like.

The network 16 may be almost any type of network. For example, in some embodiments, the network 16 may be, but is not limited to being, a version of an Internet network (e.g., exist in a TCP/IP-based network). It is conceivable that in the near future, embodiments within the present disclosure may use more advanced networking technologies.

In some embodiments, the external system 17 may optionally communicate with the host system 12. For example, in one embodiment of the system 10, the external system 17 may supply data transmissions via the network 16 to the host system 12 regarding real-time or substantially real-time events (e.g., user updates, photographic or illustration image updates, and/or user information updates). Data transmission may be through any type of communication including, but not limited to, speech, visuals, signals, textual, and/or the like. Events may include, for example, data transmissions regarding user messages or updates from a group leader, for example, initiated via the external system 17. It should be noted that the external system 17 may be the same type and construction as the user device 14.

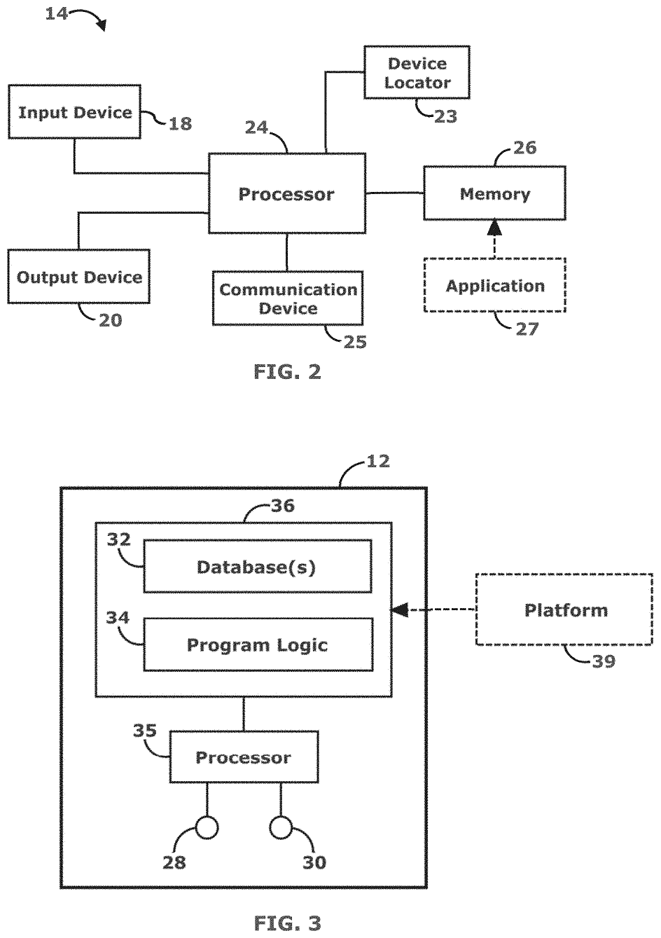

As shown in FIG. 2, the one or more user devices 14 of the system 10 may include, but are not limited to including, implementation as a personal computer, a cellular telephone, a smart phone, a network-capable television set, a tablet, a laptop computer, a desktop computer, a network-capable handheld device, a server, a digital video recorder, a wearable network-capable device, and/or the like.

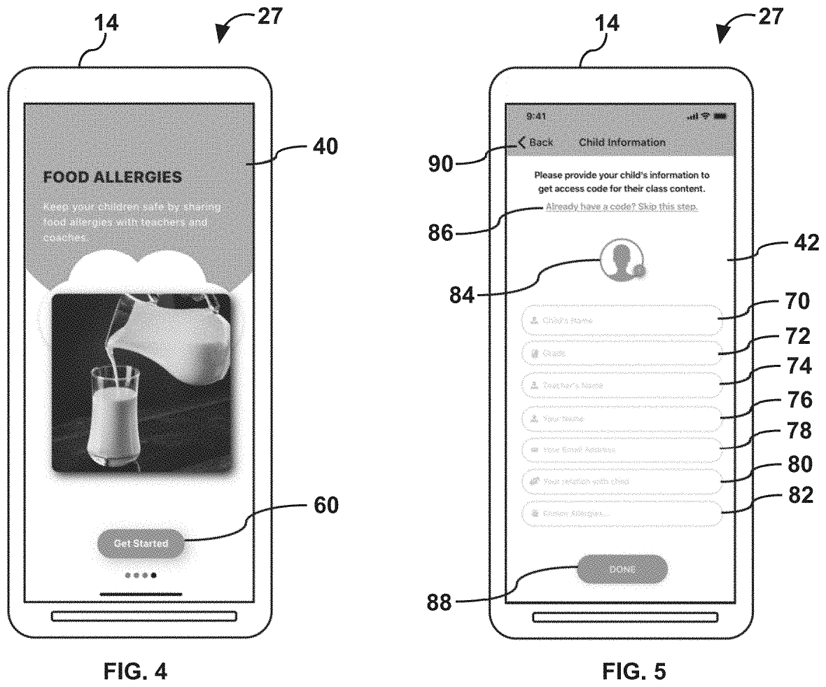

In some embodiments, the user device 14 may include one or more input devices 18 (hereinafter “input device 18”), one or more output devices 20 (hereinafter “output device 20”), a device locator 23, one or more processors 24 (hereinafter “processor 24”), one or more communication devices 25 (hereinafter “communication device 25”) capable of interfacing with the network 16, one or more non-transitory memory 26 (hereinafter “memory 26”) storing processor executable code and/or software application(s), for example including, a web browser capable of accessing a website and/or communicating information and/or data over a wireless or wired network (e.g., network 16), and/or the like. The memory 26 may also store an application 27 that, when executed by the processor 24 causes the user device 14 to automatically and without user intervention transmit or share allergy information, including an allergy and a severity of the allergy for a user or a child, for instance, associated with the user, with a predefined group of people to alert the predefined group of people about the allergy and the severity of the reaction without sharing any personal information about the user or the child. In some embodiments, the application 27 is programmed to cause the processor 24 to provide an information input screen (see FIG. 4) to the output device 20, and to receive information from the user via the input device 18. Such information can be stored either temporarily and/or permanently in the memory 26 and/or transmitted to the host system 12 via the network 16 using the communication device 25.

Embodiments of the system 10 may also be modified to use any user device 14 or future developed devices capable of communicating with the host system 12 via the network 16.

A device locator 23 may be capable of determining the position of the user device 14. For example, implementations of the device locator 23 may include, but are not limited to, a Global Positioning System (GPS) chip, software based device triangulation methods, network-based location methods such as cell tower triangulation or trilateration, the use of known-location wireless local area network (WLAN) access points using the practice known as “wardriving”, a hybrid positioning system combining two or more of the technologies listed above, or any future developed system or method of locating a device such as the user device 14.

The input device 18 may be capable of receiving information input from the user and/or processor 24, and transmitting such information to other components of the user device 14 and/or the network 16. The input device 18 may include, but are not limited to, implementation as a keyboard, touchscreen, mouse, trackball, microphone, fingerprint reader, infrared port, slide-out keyboard, flip-out keyboard, cell phone, PDA, remote control, fax machine, wearable communication device, network interface, combinations thereof, and/or the like, for example.

The output device 20 may be capable of outputting information in a form perceivable by the user and/or processor 24. For example, implementations of the output device 20 may include, but are not limited to, a computer monitor, a screen, a touchscreen, a speaker, a website, a television set, a smart phone, a PDA, a cell phone, a fax machine, a printer, a laptop computer, combinations thereof, and the like, for example. It is to be understood that in some exemplary embodiments, the input device 18 and the output device 20 may be implemented as a single device, such as, for example, a touchscreen of a computer, a tablet, or a smartphone. It is to be further understood that as used herein the term user is not limited to a human being, and may comprise, a computer, a server, a website, a processor, a network interface, a human, a user terminal, a virtual computer, combinations thereof, and/or the like, for example.

The host system 12 may be capable of interfacing and/or communicating with the user device 14 and the external system 17 via the network 16. For example, the host system 12 may be configured to interface by exchanging signals (e.g., analog, digital, optical, and/or the like) via one or more ports (e.g., physical ports or virtual ports) using a network protocol, for example. Additionally, each host system 12 may be configured to interface and/or communicate with other host systems 12 directly and/or via the network 16, such as by exchanging signals (e.g., analog, digital, optical, and/or the like) via one or more ports.

The network 16 may permit bi-directional communication of information and/or data between the host system 12, the user device 14, and/or the external system 17. The network 16 may interface with the host system 12, the user device 14, and/or the external system 17 in a variety of ways. For example, in some embodiments, the network 16 may interface by optical and/or electronic interfaces, and/or may use a plurality of network topographies and/or protocols including, but not limited to, Ethernet, TCP/IP, circuit switched path, combinations thereof, and/or the like. For example, in some embodiments, the network 16 may be implemented as the World Wide Web (or Internet), a local area network (LAN), a wide area network (WAN), a metropolitan network, a 4G network, a satellite network, a radio network, an optical network, a cable network, a public switch telephone network, an Ethernet network, combinations thereof, and the like, for example. Additionally, the network 16 may use a variety of network protocols to permit bi-directional interface and/or communication of data and/or information between the host system 12, the user device 14 and/or the external system 17.

Referring now to FIG. 3, shown therein is a diagrammatic view of an exemplary embodiment of the host system 12. In the illustrated embodiment, the host system 12 is provided with one or more databases 32 (hereinafter “database 32”), program logic 34, and one or more processors 35 (hereinafter “processor 35”). The program logic 34 and the database 32 are stored on non-transitory computer readable storage memory 36 (hereinafter “memory 36”) accessible by the processor 35 of the host system 12. It should be noted that as used herein, program logic 34 is another term for instructions which can be executed by the processor 24 or the processor 35. The database 32 can be a relational database or a non-relational database. Examples of such databases comprise, DB2®, Microsoft® Access, Microsoft® SQL Server, Oracle®, mySQL, PostgreSQL, MongoDB, Apache Cassandra, and the like. It should be understood that these examples have been provided for the purposes of illustration only and should not be construed as limiting the presently disclosed inventive concepts. The database 32 can be centralized or distributed across multiple systems.

In some embodiments, the host system 12 may comprise one or more processors 35 working together, or independently to, execute processor executable code stored on the memory 36 of the host system 12. Additionally, each host system 12 may include at least one input device 28 (hereinafter “input device 28”) and at least one output device 30 (hereinafter “output device 30”). Each element of the host system 12 may be partially or completely network-based or cloud-based, and may or may not be located in a single physical location.

The processor 35 of the host system 12 may be implemented as a single processor or multiple processors working together, or independently, to execute the program logic 34 as described herein. It is to be understood, that in certain embodiments using more than one processor 35, the processors 35 may be located remotely from one another, located in the same location, or comprising a unitary multi-core processor. The processors 35 may be capable of reading and/or executing processor executable code and/or capable of creating, manipulating, retrieving, altering, and/or storing data structures into the memory 36.

Exemplary embodiments of the processor 35 of the host system 12 may be include, but are not limited to, a digital signal processor (DSP), a central processing unit (CPU), a field programmable gate array (FPGA), a microprocessor, a multi-core processor, combinations, thereof, and/or the like, for example. The processor 35 may be capable of communicating with the memory 36 via a path (e.g., data bus). The processor 35 may be capable of communicating with the input device 28 and/or the output device 30.

The processor 35 of the host system 12 may be further capable of interfacing and/or communicating with the user device 14 and/or the external system 17 via the network 16. For example, the processor 35 may be capable of communicating via the network 16 by exchanging signals (e.g., analog, digital, optical, and/or the like) via one or more ports (e.g., physical or virtual ports) using a network protocol to provide updated information to the application 27 executed on the user device 14 such as, for instance, predefined property information such as county assessment information for a property at or near the user's current location.

The memory 36 of the host system 12 may be capable of storing processor executable code. Additionally, the memory 36 may be implemented as a conventional non-transitory memory, such as for example, random access memory (RAM), CD-ROM, a hard drive, a solid-state drive, a flash drive, a memory card, a DVD-ROM, a disk, an optical drive, combinations thereof, and/or the like, for example.

In some embodiments, the memory 36 of the host system 12 may be located in the same physical location as the host system 12, and/or one or more memory 36 may be located remotely from the host system 12. For example, the memory 36 may be located remotely from the host system 12 and communicate with the processor 35 via the network 16. Additionally, when more than one memory 36 is used, a first memory 36 may be located in the same physical location as the processor 35, and additional memory 36 may be located in a location physically remote from the processor 35. Additionally, the memory 36 may be implemented as a “cloud” non-transitory computer readable storage memory (i.e., one or more memory 36 may be partially or completely based on or accessed using the network 16).

The input device 28 of the host system 12 may transmit data to the processor 35 and may be similar to the input device 18 of the user device 14. The input device 28 may be located in the same physical location as the processor 35, or located remotely and/or partially or completely network-based. The output device 30 of the host system 12 may transmit information from the processor 35 to a user, and may be similar to the output device 20 of the user device 14. The output device 30 may be located with the processor 24, or located remotely and/or partially or completely network-based.

The memory 36 of the host system 12 may store processor executable code and/or information comprising the database 32 and program logic 34. In some embodiments, the processor executable code may be stored as a data structure, such as the database 32 and/or data table, for example, or in non-data structure format such as in a non-compiled text file.

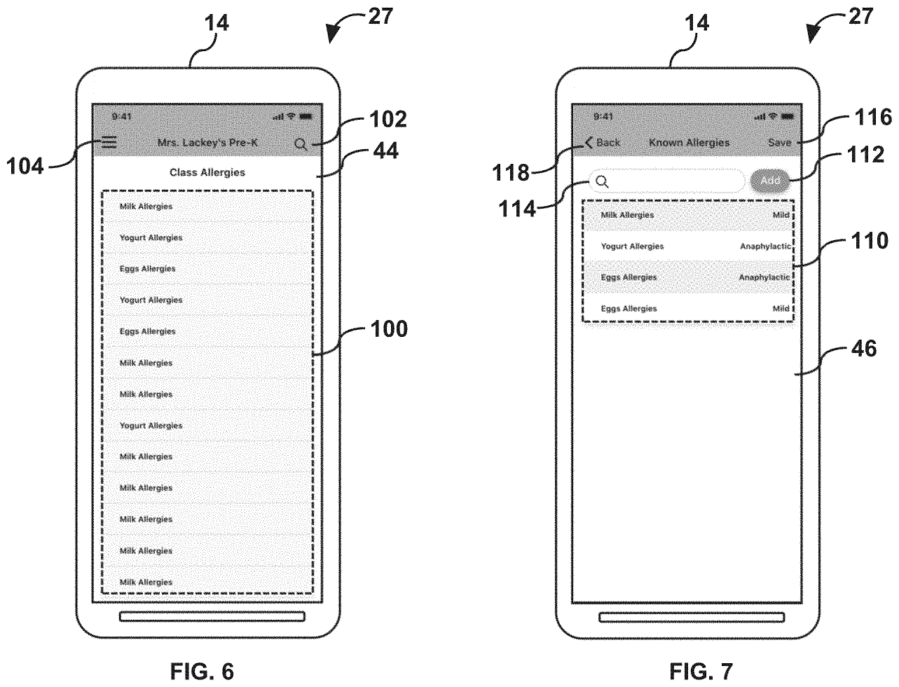

As illustrated in FIGS. 4-11, the system 10 for anonymously sharing allergy information may include the application 27 executed by the processor 24 of the user device 14 that is capable of communicating with the host system 12 via the network 16. The system 10 may include a separate program, application or “app”, or a widget, each of which may correspond to instructions stored in the memory 26 of the user device 14 for execution by the processor 24 of the user device 14. Alternately, the system 10 may include instructions stored in the memory 36 of the host system 12 for execution by the processor 35 of the host system 12 with results sent via the network 16 to be displayed on the output device 20 of the user device 14.

The instructions of the application 27, when executed by the processor 24 of the user device 14, cause the user device 14 to perform certain tasks. For example, such tasks may include displaying content such as a get started screen 40, information input screen 42, a group allergy list screen 44, an allergy severity screen 46, an allergy severity input screen 48, a group members screen 50, a menu screen 52, and a user profile screen 54. As illustrated in FIGS. 4-8, the get started screen 40, the information input screen 42, the group allergy list screen 44, the allergy severity screen 46, the allergy severity input screen 48, the group members screen 50, the menu screen 52, and the user profile screen 54 are shown as such screens may appear on the output device 20 of the user device 14, where the user device 14 is a smart phone or tablet such as an Apple® iPhone® or iPad®. The application 27 may be implemented for use on other types of user devices 14 including, but not limited to, other mobile devices, personal computers, or laptop computers, with appropriate storage and processing capacity and internet or network connectivity. The user of the system 10 may interact via user interface implementations of the user device 14 such as, for instance, by using a touchscreen of a smart phone or tablet. In some embodiments of the system 10, certain viewable screens of the application 27 may be designed to automatically switch from portrait to landscape presentation on the output device 20 of the user device 14 depending on the current orientation of the user device 14 being utilized. It should be noted that such functionality is optional, and has no adverse impact on the functionality of the application 27.

The application 27 may be provided with a login screen which may have fields designed for input from the user associated with the user device 14, for instance, a username field, and a password field to authenticate the user. After accepting input from the user, the application 27 may authenticate the user, such as by verifying the username and password entered by the user. Authentication may take place on the user device 14 or, in some embodiments, the application 27 may communicate with the host system 12 to authenticate the user information. In some embodiments, the application 27 may be programmed to accept an alternative login function which provides login functionality and allows the user to login to the system 10 via other authentication or verification methods such as through online social networks, such as Linkedin.com or Facebook.com. Some embodiments of the system 10 may also allow a new user to register their information from the login screen of the application 27.

After the user logs in or is otherwise granted access to the application 27, the logic may branch from the login screen to the get started screen 40, for example. Shown in FIG. 4 is an exemplary get started screen 40 of the application 27. The get started screen 40 may be provided with a selection section or area such as a get started button 60 configured to accept input from the user indicating a desire to start inputting allergy information.

Shown in FIG. 5 is an exemplary information screen 42 of the application 27. The information screen 42 may be provided with fields designed to accept input from the user, such as a name field 70 for identifying a person with an allergy, a group field 72, a group leader field 74, a user's name field 76, a contact information field 78, a relationship field 80, and/or an allergy input field 82. The information screen 42 of the application 27 may also be provided with fields or input selection areas, such as a picture area 84 and a skip button 86. The picture area 84 may be programmed to accept input from the user indicating a desire to add or change a picture to help in identifying the person with the allergy.

In some embodiments, some or all of the information on the information screen 42 may be input by an administrator or other user. For instance, when the application 27 is being used by a school, the school may use registration information to input certain information and then send a code or other identifier to a parent of a student that allows the parent to access the system 10 and correct or complete any missing information. In such embodiments or methods, the user may select the skip button 86 which may access a code screen that allows the user to input the supplied code or other identifier to access the already completed information and allow them to change and/or supplement the information to ensure that it is correct.

The information screen 42 of the application 27 may also be provided with buttons such as a done button 88 and a back button 90. The done button 88 may be programmed to save the input information and navigate to another screen in the application 27. The back button 90 may be programmed to allow the user to navigate to a previous screen of the application 27.

It should be noted that the exemplary information screen 42 illustrates a use case where the user is a parent that is adding information regarding their child who has an allergy. In such a case, the person with the allergy is not the user, but a dependent of the user. However, it should be understood that in some embodiments, the user may be the person with the allergy in which case the information screen 42 may include other fields designed to accept input from the user. In either case, information regarding the person having the allergy, the allergy, and a reaction to the allergy are associated with a user profile of the user and stored as part of the application 27 or in the database 32 on the host system 12, for instance.

The group allergy list screen 44 may allow users that are members of a group to view a list of allergies that group members have. For instance, the group allergy list screen 44 may be provided with an allergy list section 100 that contains a list of all of the allergies for members of a selected group. For instance, the illustrated group allergy list screen 44 is for a school class group labeled “Mrs. Lackey's Pre-K.” The application 27 and or the program logic 34 may be programmed to associate information input into a name field 70 identifying the person with an allergy with the group that is selected by a user or input by a group leader or administrator, for instance. Once the information identifying the person with an allergy has been associated with the group, information regarding the person's known allergies may be included in the allergy list section 100.

The group allergy list screen 44 may also be provided with a search button 102 and a menu button 104. The search button 102 may be programmed to allow the user to search through entries in the allergy list section 100. The menu button 104 may be programmed to allow the user to navigate the various screens and/or sections of the application 27.

The group allergy list screen 44 may be made available to group members (e.g., classmates, parents, teammates, coworkers, etc.) so they may see allergies that other group members have. As illustrated in the exemplary group allergy list screen 44, these allergies are shared anonymously so the group members are aware of the allergies, but not who has the allergy. This way, unintended allergic reactions can be avoided without stigmatizing the person with the allergy.

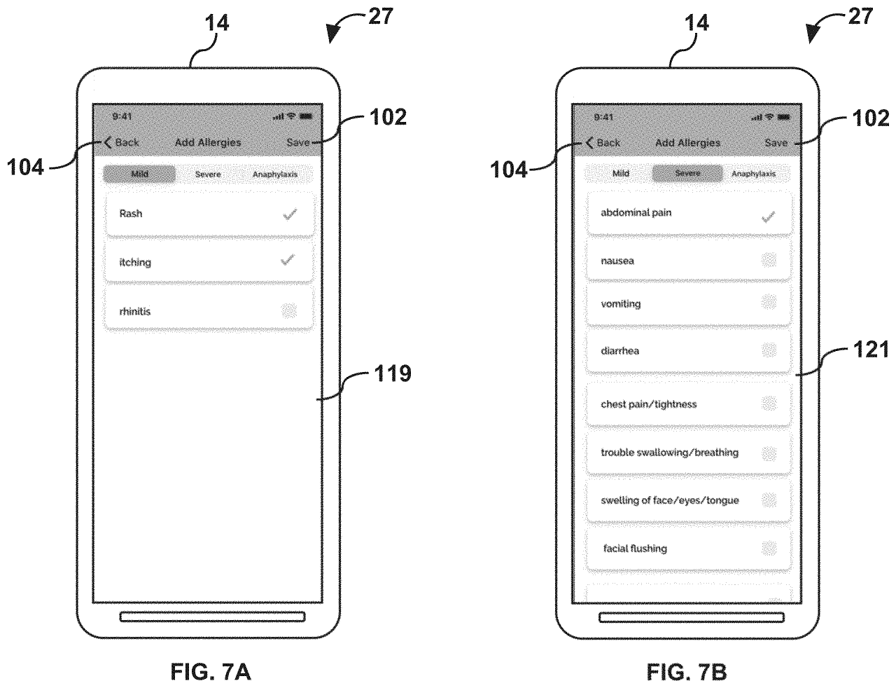

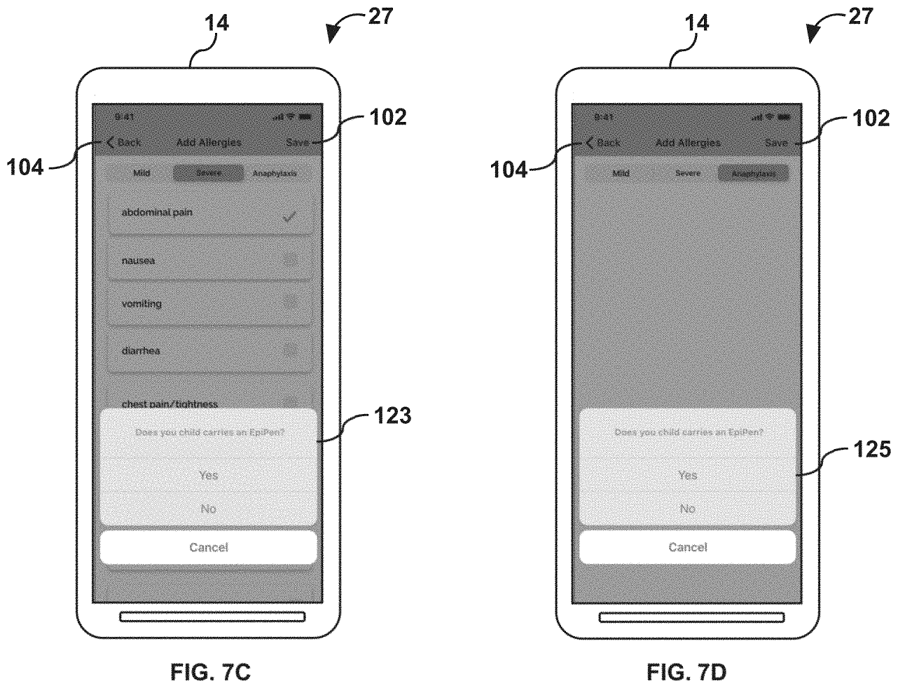

While the group allergy list screen 44 is shown only listing allergies in the allergy list section 100, in some embodiments of the system 10, the allergy list section 100 may include other information such as a severity of an allergic reaction to the allergy (similar to the list illustrated in FIG. 7 and labeled allergy severity section 110) and/or possible symptoms of an allergic reaction to the allergy. This way, group members may be aware of the severity of a reaction to an allergy and/or symptoms to be aware of, without knowing which user (or child) in the group has the allergy.

The allergy severity screen 46 may be provided with an allergy severity section 110 programmed to display a list of known allergies associated with a group and a severity of a reaction to the allergy. Three exemplary options for severity may be mild, severe, and anaphylaxis (which may also or in the alternative be labeled “life-threatening”). The application 27 may include short descriptions associated with each severity type or a list of exemplary symptoms to help users in selection. For instance, as illustrated in FIG. 7A, symptoms of a mild reaction may include rash, itching, and rhinitis displayed on a mild symptom screen 119. The mild symptoms screen 119 may include selectable areas that allow the user to select which symptoms are associated with the allergy.

FIG. 7B illustrates an exemplary severe symptom screen 121. Exemplary symptoms of a severe reaction may include abdominal pain, nausea, vomiting, diarrhea, chest pain/tightness, trouble swallowing/breathing, swelling of face/eyes/tongue, facial flushing, dizziness, wheezing, heart palpitations. The severe symptom screen 121 may include selectable areas that allow the user to select which symptoms are associated with the allergy.

As shown in FIG. 7C, the application 27 may also be provided with a pop-up screen 123 that is provided with selectable regions that allow the user to select whether or not the person (child) with the allergy carries an epinephrine autoinjector (such as an EpiPen) to treat their severe reaction.

As shown in FIG. 7D, the application 27 may also be provided with an anaphylaxis screen 125 that may include selectable regions that allow the user to select whether or not the person (child) with the allergy carries an epinephrine autoinjector (such as an EpiPen) to treat their anaphylactic reaction. Symptoms of anaphylaxis include difficulty breathing, hives or other skin changes, and sudden swelling of the face, lips or throat.

The severity of the reaction to the allergen allows group members to avoid items that may be harmful or uncomfortable for other members of the group and to gauge risk when considering activities. For instance, when a parent of a child that is a member of a team is asked to bring a snack to a game, the parent may use the application 27 to view the allergy severity screen 46 to see what allergies they need to be aware of and also the severity of any allergic reaction. In the exemplary allergy severity screen 46, the parent would see that members of the team have allergies to milk, yogurt, and eggs and the severity of a reaction to yogurt and eggs is “anaphylactic.” As a result, the parent would know not only to be extremely careful not to bring a snack that contained yogurt or eggs but to also avoid milk products as well.

Returning now to FIG. 7, the allergy severity screen 46 may also be provided with an add button 112, a search section 114, a save button 116, and a back button 118. The user may use the back button 118 to navigate to a previous screen of the application 27.

The add button 112 may be programmed to allow a user with appropriate permissions, such as a group leader, to manually input an allergy and or a severity of reaction to be associated with the group. For instance, if a new member joins the group and their information has not yet been added to the system 10, the user with appropriate permissions may input their allergy and severity of reaction manually so that the list reflects all of the allergies and severity of reactions for the group.

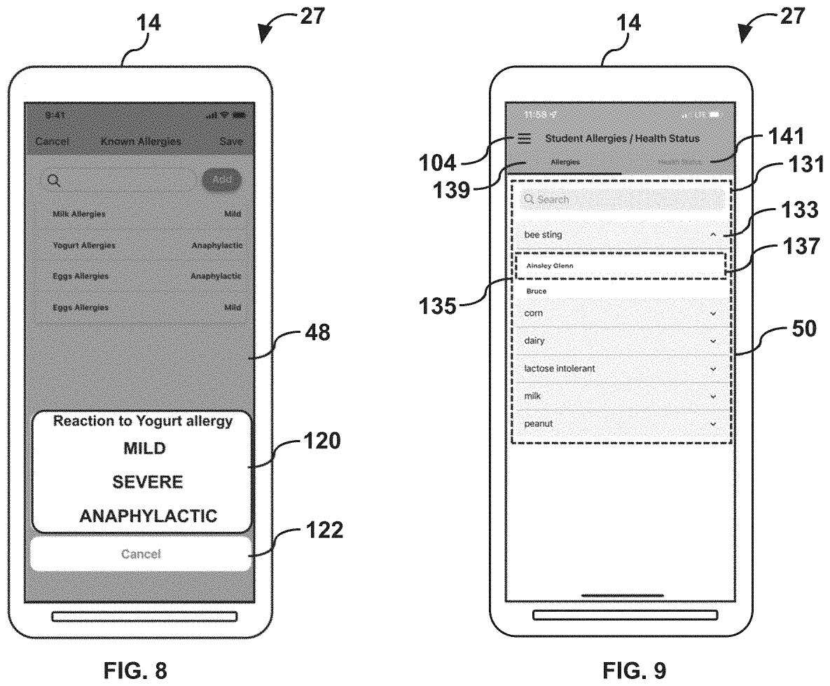

The search section 114 may be programmed to accept input from the user to allow the user to search a list of allergies to allow easy addition to the allergy severity section 110. Once a new allergy has been selected from the search list or manually input by the user, the application 27 may be programmed to display the allergy severity input screen 48 to allow the user to select a severity of a reaction to the allergen. For instance, a list of severities may be displayed in a severity section 120 (FIG. 8). In the exemplary severity section 120, the options are mild, severe, and anaphylactic. It should be noted, however, that other options may be used including changing the name of the severities, expanding the list of severities to include other options, etc.

In the exemplary application 27, once the user selects a severity of a reaction to the selected allergy, the application 27 may be programmed to display the allergy severity screen 46 where the user may review the addition of the new allergy and severity of reaction before selecting the save button 116. If the user does not wish to continue adding the new allergy and severity of reaction, the user may select a cancel button 122 on the allergy severity input screen 48 which will take them back to the allergy severity screen 46 without adding the new allergy or the severity of reaction.

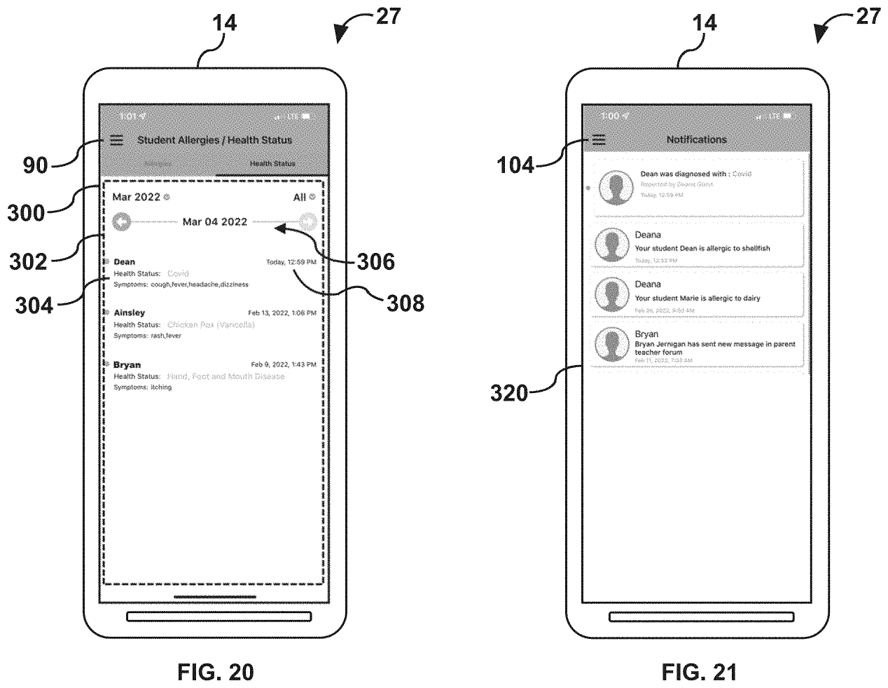

As shown in FIG. 9, the group members screen 50 may be provided with a list section 131 comprising a list of allergies associated with members of the group. In the illustrated example, the allergies include bee sting, corn, dairy, lactose intolerant, milk, and peanut. Each allergy may have an associated button 133 (only one of which is labeled in FIG. 9, for clarity). The user may select an allergy in the list section 131 by selecting the associated button 133 which may expand a name section 135 illustrating all members of the group who have the selected allergy. This way, a user such as a teacher, may view a list of allergies associated with members of the group and further view specific members of the group who have the associated allergy. The group members screen may be provided with an allergy section 139 (displayed in FIG. 9) and a health status section 141 (see FIG. 20).

Each name in the name section 135 may have an associated selectable area that functions as a member button 137 (only one of which is shown in FIG. 9 for clarity). When selected, the member button 137 may be programmed to display more information about the associated name. For instance, the more information could include a severity of reaction associated with each of the known allergies, and, in the instance of an anaphylactic severity, the more information may include whether or not the student carries an epinephrin pen (such as an EpiPen) and where the epinephrin pen is located (e.g., in a front pocket of their red backpack). This way, teachers or school administrators (in other words group leaders or those with administrator access) are aware of the severity of reactions as well as any possible treatments that may be available.

In some embodiments, the system 10 may be programmed to automatically alert a predetermined group of users (e.g., teacher, coach, boss, etc.) when a member of the group has a possible severe or anaphylactic reaction to an allergen as well as any treatment that may be available. This way, the predetermined group of users may be prepared to react and/or know to review the information associated with the user in the application 27 to determine if there is a treatment available. In some embodiments, the other information that is displayed when the member button 137 is selected may include emergency contact information such as a parent's phone number, a doctor's phone number, or a specialist's phone number, for instance.

The more information may also include information relating to symptoms of an allergic reaction associated with each allergy that may help the user identify an allergic reaction. For instance, for a user named Henry who has three known allergies to milk, peanuts, and eggs, the more information may include additional information such as: 1) Mild Milk Allergy (Itching); 2) Severe Peanut Allergy (Nausea, Dizziness); and 3) Anaphylactic Reaction to Eggs (carries an EpiPen). Such information or similar information could be included for each user (or the child of a user) with known allergies.

It should be noted that the group members screen 50 is not available to all users. The group members screen 50 contains personally identifiable information that is only made available to certain users such as a group leader (teacher, coach, boss, etc.) or those with administrator access. This way, the identity of the user (or child) with the allergy is protected from the group at large, but more in-depth information such as name, responsible party (such as a parent), emergency contact information, reaction symptoms, treatment options, etc., are made available to certain individuals in the group so that a possible allergic reaction can be handled as safely as possible and those that need to be notified can be.

It should be noted that in some embodiments a group members screen may be provided with a list section comprising a list of the users in a group who have allergies. For example, the group is a class and the listed users (or children of users) are students. Each of the students listed may have an associated button which allows the user to view more information about the student. For instance, the more information could include a severity of reaction associated with each of the known allergies, and, in the instance of an anaphylactic severity, the more information may include whether or not the student carries an epinephrin pen (such as an EpiPen) and where the epinephrin pen is located (e.g., in a front pocket of their red backpack). This way, teachers or school administrators (in other words group leaders or those with administrator access) are aware of the severity of reactions as well as any possible treatments that may be available.

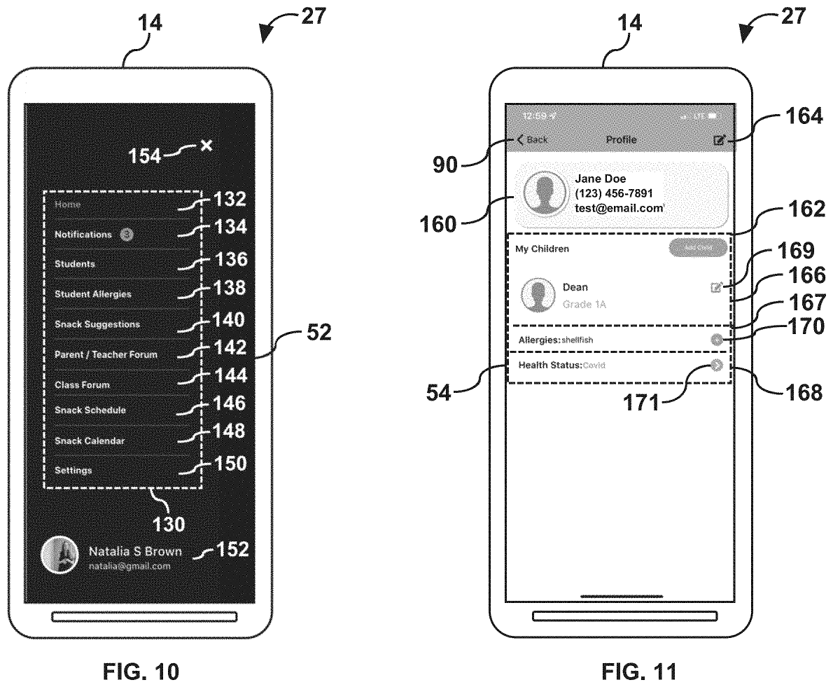

As shown in FIG. 10, the menu screen 52 may be provided with a menu section 130 having navigation buttons such as a home button 132, a notifications button 134, a group members button 136, a group members allergy button 138, a snack suggestions button 140, a responsible party button 142, a group forum button 144, a snack calendar 146, and a settings button 150. The menu screen 52 may also be provided with a user identification section 152 and a close button 154. The navigation buttons in the menu section 130 on the menu screen 52 allow the user to access the various aspects and screens of the application 27. The menu screen 52 may be displayed and or accessed when the user selects the menu button 104 as illustrated as part of the group allergy list screen 44, for instance.

In one embodiment of the application 27, the settings button 150 allows the user to personalize the look, feel, and function of the application 27, however, it will be understood that such functionality is optional, and has no adverse impact on the functionality of the application 27.

It should be noted that the navigation buttons shown as part of the menu section 130 are for the purposes of illustration only. The exemplary menu screen 52 in FIG. 10 has optional buttons for a leader of a group such as a teacher (in this example, Natalie S. Brown). Group leaders will have access to information not available to all users such as a list of names of students (group members) and a list of the names of students with known allergies associated with each student. Therefore, a person of skill in the art will appreciate that the menu section 130 of the menu screen 52 may be different for different users and the illustrated navigation buttons are for the purposes of illustration only. Further, the labels of the navigation buttons on the exemplary menu screen 52 may be different in different embodiments. For example, the exemplary menu screen 52 in FIG. 10 is for a school group. Therefore, the labels of the navigation buttons are school-centric (e.g., students, student allergies, parent/teacher forum, class forum). However, it should be understood that the labels of the navigation buttons may be customized or changed in different embodiments. For example, where the group is a sports team, the group members button 136 may be labeled “Athletes” and the group members allergy button 138 may be labeled “Athlete Allergies.” Therefore, it should be recognized that these labels do not change the functioning of the system 10 and are included not by way of limitation but for the purposes of describing the application 27.

As shown in FIG. 11, the user profile screen 54 allows the user to view and/or edit information associated with their user profile. The user profile screen 54 may be provided with a user information section 160 and an allergy detail section 162.

The exemplary user information section 160 displays information input by the user and/or a group leader or administrator that is associated with the user profile. Exemplary information may include a picture of, or associated with, the user as well as the user's name, address, and contact information such as a phone number and/or email address. The user information section 160 may be provided with an edit button 164 that allows the user to change the information in the user information section 160 if the information changes or is incorrect. It should be noted that changing the information in the user information section 160 may change the information in the user profile stored in the application 27 or on the host system 12 in the database 32, for instance.

The allergy detail section 162 may be provided with a dependent detail section 166. Each dependent detail section 166 may be associated with a person having an allergy. In the illustrated example, the user (Jane Doe) is the parent of Dean who has an associated allergy and an associated health status. It will be recognized by a person of skill in the art that the number of dependent detail sections 166 may be different for different users. Further, in a case where the user is the person having an allergy, it will be the user's allergy information that is displayed in the dependent detail section 166.

Each dependent detail section 166 may be provided with an allergy section 167 that displays all allergies associated with the dependent, a health status section 168 that displays a current health status of the dependent, and an edit button 169 that allows the user to edit the information in the dependent detail sections 166. Further, while in some embodiments the system 10 may be programmed to automatically update group association for each user (or child/dependent), in some embodiments, the user may add or change the groups they are associated with in the user detail sections 166.

The allergy section 167 may be provided with an add allergy button 170 that may be programmed to allow a user to add a new allergy associated with the dependent displayed in the dependent detail section 166. For instance, if Dean were to develop a new allergy, the user may select the add allergy button 170 and add the new allergy.

The health status section 168 may be provided with a change button 171 that allows the user to change the health status associated with the dependent displayed in the dependent detail section 166. The change button 171 may be programmed to cause the user device 14 to display a new screen that allows the user to change the health status of the associated dependent as will be described further herein.

It should be noted that not all of the information that may be associated with each user (or child/dependent) having an allergy is displayed in the exemplary user detail sections 166. Other information, as described herein, may be associated with each user (or child/dependent) having an allergy and may be displayed and/or available for editing in the user detail sections 166 and the information that is displayed should in no way be considered limiting.

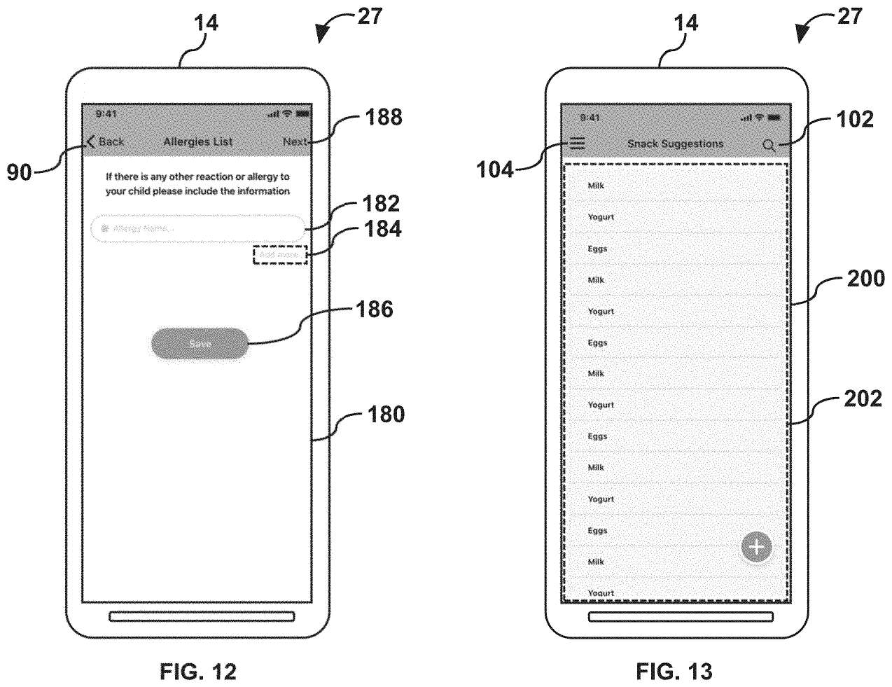

Referring now to FIG. 12, an exemplary add reaction screen 180 is shown. The add reaction screen 180 may be provided with an add region 182 that is programmed to accept input from the user indicative of a reaction to an allergy. For instance, when a reaction or symptom is not listed on one of the mild symptom screen 119 or severe symptom screen 121, the user may use the add reaction screen 180 to add a new symptom that will be associated with the person having the allergy.

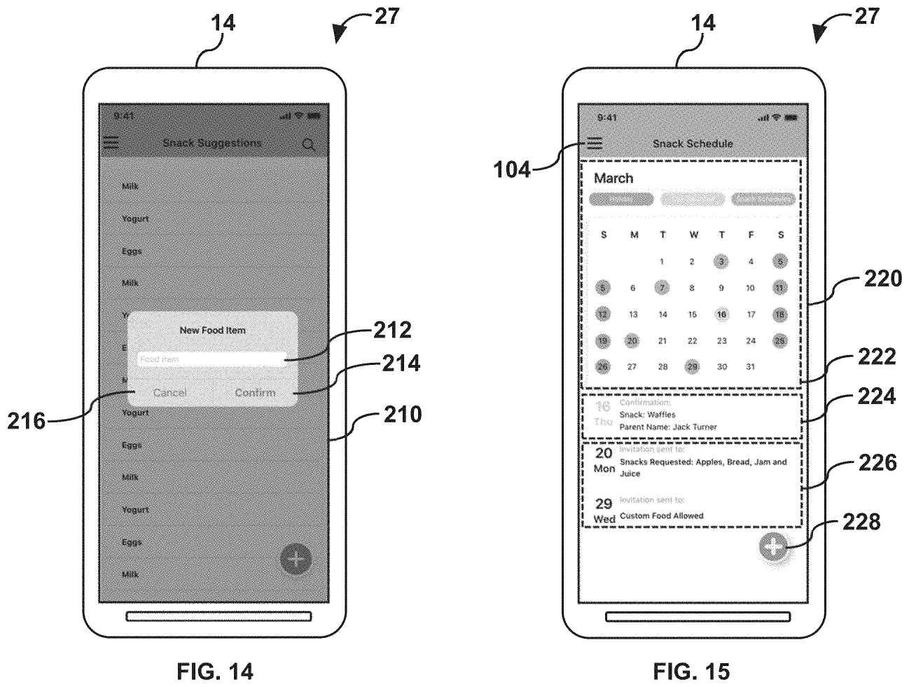

Referring now to FIG. 13, an exemplary snack suggestion screen 200 is shown. The snack suggestion screen 200 may be provided with a list region 202, search button 102, and menu button 104. The list region 202 may be populated with a list of snacks that are appropriate for a given group taking into account all of the known allergies that have been input into the system 10 associated with members of the group. The snack suggestions in the list region 202 may be input by the leader of the group or an administrator using a snack suggestion screen 210 as shown in FIG. 14.

The exemplary add snack suggestion screen 210 shown in FIG. 14 is provided with a suggestion input region 212, a confirm button 214, and a cancel button 216. The suggestion input region 212 may be configured to accept input from the user indicative of an appropriate food item for the group that takes into account the known allergies of the group. In some embodiments, the snack suggestion screen 210 may allow the user to input food preferences as well. In such an embodiment, the snack suggestion screen 210 may be programmed to allow the user to differentiate between snacks that are suggested to avoid known allergies and those that are suggested as a preference.

Referring now to FIG. 15, an exemplary snack schedule screen 220 is shown. The snack schedule screen 220 may be provided with a calendar section 222, a confirmed snack date section 224, an unconfirmed snack date section 226, an add button 228, and menu button 104.

The calendar section 220 may display dates from a given time frame, such as a month, for example (March is shown for the purposes of illustration). In the calendar section 220, dates with confirmed and/or unconfirmed snack dates may be shown. To differentiate between confirmed and/or unconfirmed snack dates, the dates may be shown with different colors or shades, as illustrated in FIG. 15. However, it should be noted that other ways of showing the difference may be used such as different shapes around the dates, different colors for the date number, or different fonts, for instance.

The confirmed snack date section 224 may display snack dates that have been confirmed. The confirmed snack date section 224 may further display a snack (waffles) that will be provided and a person who will provide the snack (Jack Turner). While the confirmed snack date section 224 is illustrated showing only one confirmed date, it should be understood that the confirmed snack date section 224 may display more than one confirmed date with the associated information.

The unconfirmed snack date section 226 may display snack dates where an invitation has been sent but not yet confirmed. The unconfirmed snack date section 224 may display snacks that have been requested (apples, bread, jam, and juice) and the date on which they have been requested (20 Monday), for instance. While the unconfirmed snack date section 226 is illustrated showing two unconfirmed dates, it should be understood that the unconfirmed snack date section 226 may display one or unconfirmed dates with the associated information.

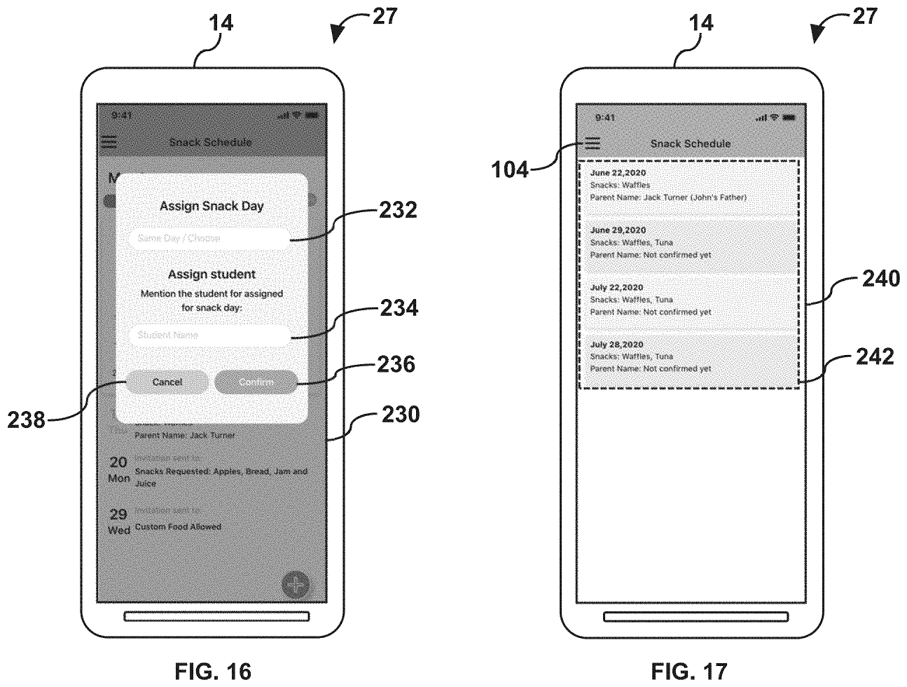

To add a new snack date, the application 27 may be provided with an assign snack date screen 230 as illustrated in FIG. 16. The assign snack date screen 230 may be provided with a date selection section 232, an assign user section 234, a confirm button 236, and a cancel button 238.

The date selection section 232 may be configured to accept input from the user to select a date for a desired snack day. In some embodiments, the date for the desired snack day may be input as text. In some embodiments, the application 27 may be configured to pull up a calendar that allows the user to select the date for the desired snack day.

The assign user section 234 may be configured to accept input from the user to select a user (or parent) to assign the desired snack day to. In some embodiments, the user for the desired snack day may be input as text. In some embodiments, the application 27 may be configured to access a list of users associated with the group when the user selects the assign user section 234 and display them as a selectable list.

Once the user has input the date for the desired snack and selected a user to assign the desired snack day, the user may select the confirm button 236 to save the inputs. If the user wishes to discard the changes, the user may select the cancel button 238.

In some embodiments, the application 27 may be programmed to allow access to the assign snack date screen 230 to a predetermined group of users such as the leader of the group and the administrator, for instance.

Referring now to FIG. 17, the application 27 may be provided with a snack schedule screen 240. The snack schedule screen 240 may be provided with a snack schedule section 242 configured to display confirmed and/or unconfirmed snack dates. In the illustrated embodiment, the snack schedule section 242 is configured to display both confirmed (e.g., June 22, 2020) and unconfirmed snack dates (e.g., June 29, 22, and 28, 2020). In the illustrated embodiment, the confirmed snack date displays the date, a snack that will be provided, and a user (or associated parent) who has agreed to supply the snack. The unconfirmed snack dates display a desired snack date and a list of suggested snacks.

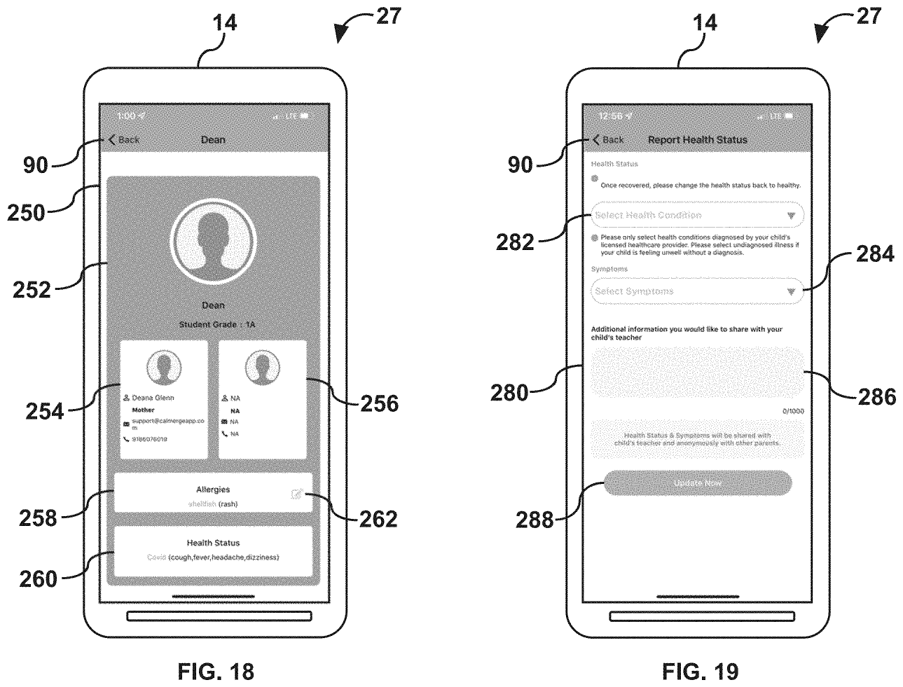

Referring now to FIG. 18, the application 27 may be provided with a child information screen 250. The child information screen 250 may be provided with a child information section 252, a first guardian section 254, a second guardian section 256, an allergy section 258, and a health status section 260. In some embodiments, the allergy section 258 of the child information screen 250 may be provided with an edit button 262.

The child information screen 250 may display information input on different screens such as the information input screen 42 described above. The child information screen 250 may be accessed by select users such as group leaders, teachers, coaches, and the like when information such as contact information contained in the first guardian section 254 and/or the second guardian section 256 is needed.

While the application 27 has been described herein as being used to disseminate anonymous allergy information, it should be noted that the application 27 may be used to disseminate other anonymous information as well. For instance, the application 27 may be programmed to disseminate information about food sensitivities or intolerances such as those to gluten, dairy, caffeine, salicylates, amines, sulfites, and fructose. It should be noted that the foregoing is a non-exclusive list that is provided by way of example only and is not meant to be limiting.

The application 27 may also be used to disseminate other health related information anonymously to a group. For instance, the application 27 may be used alert the group when a member of the group has been diagnosed with a disease such as coronavirus disease 2019 (COVID-19), chicken pox, a common cold, conjunctivitis, parvovirus B19 (fifth's disease), hand, foot, and mouth disease, hepatitis A, hepatitis B, impetigo, influenza, lice, measles, mononucleosis, mumps, pinworm, ringworm, exanthem subitum (roseola), rubella, scabies, scarlet fever, strep throat, pertussis (whooping cough), and fever of unknown origin. It should be noted that the foregoing is a non-exclusive list that is provided by way of example only and is not meant to be limiting. The sharing of health information is voluntary. Sharing the health information anonymously in a timely manner allows other members of the group to start monitoring for symptoms to help prevent the spread of communicable diseases. Because the health information is shared anonymously, the person who has the condition is not excluded or stigmatized.

Referring now to FIG. 19, health related information may be entered on a health report screen 280 of the application 27. The health report screen 280 may be provided with a health condition field 282, a symptoms field 284, an additional information field 286, and an update button 288.

The health condition field 282 and the symptoms field 284 of the health report screen 280 may be provided in the form of drop-down lists that allows the user to select from a predetermined list of health conditions and symptoms. The symptoms field 284 of the health report screen 280 may also allow the user to enter new information that is not included in the drop-down lists using the input device 18 of the user device 14.

In embodiments of the application 27 where other health information is shared anonymously, the application 27 may be programmed to send an alert to all members of the group that someone in the group was diagnosed with a disease. The alert may be in the form of a pop-up in the application 27, an email, an SMS text message, a phone call, or the like. The application 27 may be programmed to send the alert automatically and without user intervention within a predetermined amount of time. The predetermined amount of time may be one (1) second, one (1) minute, ten (10) minutes, or one (1) hour, for instance. It should be noted that the foregoing is a non-exclusive list or times that is provided by way of example only and is not meant to be limiting.

Referring now to FIG. 20, the application 27 may be provided with a health status screen 300. The health status screen 300 may be provided with a list section 302 that shows a health status 304 (only one of which is numbered for clarity in the figure) for select group members who have a predetermined status(es) on selected given date 306. The predetermined statuses may be any status other than healthy, for instance, or a sub-group of health statuses that may be selected by the group leader. The health status 304 may be provided with a date 308 that represents the date the health status was reported. That way, the user such as the group leader reviewing the health status screen 300 may be able to estimate when the group member may recover and/or return to class, for instance.

Referring now to FIG. 21, the application 27 may be provided with a notifications screen 320. The notifications screen 320 provides a place where notifications for the application 27 may be gathered and viewed. Notifications may include a change in health status, a new allergy, a message, etc. Notifications may be shown based on a status, date, time, etc.

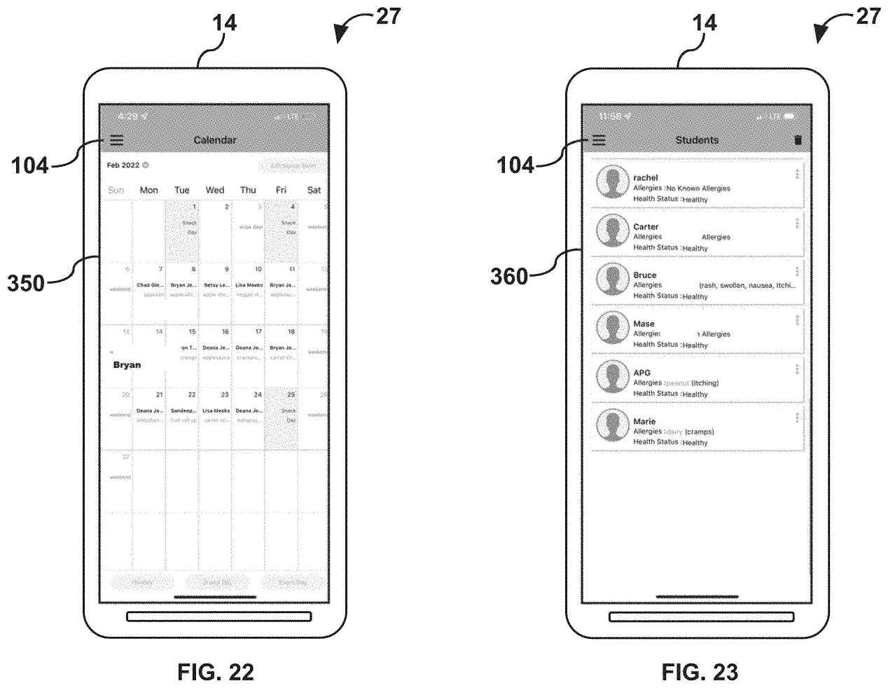

Referring now to FIG. 22, a calendar screen 350 is illustrated in accordance with one embodiment of the application 27.

Referring now to FIG. 23, a student list screen 360 is illustrated in accordance with one embodiment of the application 27.

The following is a list of illustrative embodiments and is intended to complement, rather than displace or supersede, the previous descriptions.

1. A method, comprising:

establishing communication between a host system having a processor and a plurality of user devices each having an input device, an output device, and a communication device;

accepting, via the input device of a first one of the plurality of user devices associated with a first user, input comprising user information, allergy information including an allergy and a severity of the allergy, and at least one group associated with the first user;

storing, at the host system, the user information, allergy information, and the at least one group in a user profile of the first user;

automatically, without user intervention, generating an alert, the alert containing information indicative of the allergy and the severity of the allergy but none of the user information; and

automatically, without user intervention, transmitting the alert to at least one second one of the plurality of user devices associated with at least one second member of the at least one group associated with the first user.

2. The exemplary method of illustrative embodiment 1, wherein the alert is a first alert, the method further comprising the steps of:

generating a second alert, the second alert comprising information indicative of the allergy, the severity of the allergy, and the user information; and

transmitting the second alert to a third one of the plurality of user devices associated with a leader of the at least one group associated with the first user.

3. The exemplary method of illustrative embodiment 1, wherein the allergy information further includes a treatment for an allergic reaction associated with the allergy.

4. The exemplary method of illustrative embodiment 3, wherein the alert is a first alert, the method further comprising the steps of:

generating a second alert, the second alert comprising information indicative of the allergy, the severity of the allergy, the treatment for an allergic reaction associated with the allergy, and the user information; and

transmitting the second alert to a third one of the plurality of user devices associated with a leader of the at least one group associated with the first user.

5. The exemplary method of illustrative embodiment 1, further comprising the steps of:

generating and storing information indicative of a date and time of at least one event associated with the at least one group associated with the first user; and

re-transmitting, a predetermined amount of time before the at least one event, the alert containing information indicative of the allergy and the severity of the allergy, but none of the user information, to the at least one second one of the plurality of user devices associated with at least one second member of the at least one group associated with the first user.

6. The exemplary method of illustrative embodiment 1, further comprising the step of generating and storing information indicative of a date and time of at least one event for the at least one group associated with the first user, wherein the alert is generated and transmitted a predetermined amount time before the at least one event.

7. The exemplary method of illustrative embodiment 6, further comprising the step of associating at least one second user with the at least one event, wherein the alert is generated and transmitted a predetermined time before the at least one event only to the at least one second user.

8. A system for automatically distributing allergy information, comprising:

a host system having a non-transitory storage media storing information indicative of registered users associated with a group,

a plurality of user devices, at least one of the user devices being a first user device and at least one of the user devices being a second user device different from the first user device;

wherein the host system is programmed to receive information from the first user device over a network indicative of allergy information from a first one of the registered users and store the allergy information within a database stored on the non-transitory storage media, the allergy information comprising an allergy, an indication of a severity of a reaction to the allergy, and a treatment for an allergic reaction to the allergy; and

the host system further programmed to automatically and without user intervention disseminate an alert comprising the allergy and the indication of a severity of a reaction to the allergy over the network to at least one of the second user devices, the second user devices being associated with a defined group of the registered users associated with the group, the defined group of the registered users being second users.

9. The system for automatically distributing allergy information of illustrative embodiment 8, wherein the alert is a first alert and the host system is further programmed to:

generate a second alert, the second alert comprising information indicative of the allergy, the severity of the allergy, and the user information; and

transmit the second alert to a third one of the plurality of user devices associated with a leader of at least one group associated with the first user and the second user.

10. The system for automatically distributing allergy information of illustrative embodiment 8, wherein the allergy information further includes a treatment for an allergic reaction associated with the allergy.

11. The system for automatically distributing allergy information of illustrative embodiment 10, wherein the alert is a first alert and the host system is further programmed to:

generate a second alert, the second alert comprising information indicative of the allergy, the severity of the allergy, the treatment for an allergic reaction associated with the allergy, and the user information; and

transmit the second alert to a third one of the plurality of user devices associated with a leader of at least one group associated with the first user.

12. The system for automatically distributing allergy information of illustrative embodiment 8, wherein the alert is a first alert and the host system is further programmed to:

generating and storing information indicative of a date and time of at least one event associated with the at least one group associated with the first user; and

re-transmitting, a predetermined amount of time before the at least one event, the alert containing information indicative of the allergy and the severity of the allergy, but none of the user information, to the at least one second one of the plurality of user devices associated with at least one second member of the at least one group associated with the first user.

13. The system for automatically distributing allergy information of illustrative embodiment 8, wherein the host system is further programmed to generate and store information indicative of a date and time of at least one event for at least one group associated with the first user; and

wherein the host system is programmed to transmit the alert a predetermined amount time before the date and time of the at least one event.

14. The system for automatically distributing allergy information of illustrative embodiment 13, wherein the host system is further programmed to associate at least one second user with the at least one event, wherein the alert is generated and transmitted a predetermined time before the at least one event only to the at least one second user.

15. A system, comprising:

a host system comprising:
a first processor and at least one first non-transitory computer readable medium storing a first set of instructions that when executed by the first processor cause the first processor to obtain and record in a database event information associated with a group, the event information having parameters indicative of a date and time of at least one event that is to be attended by members of the group;

a user device comprising:
a second processor, an input device, an output device, a communication device, and at least one second non-transitory computer readable medium storing a second set of instructions that when executed by the second processor cause the second processor to obtain user information associated with a user of the user device and allergy information associated with the user, the allergy information comprising an allergy, a severity of the allergy, and a treatment for an allergic reaction associated with the allergy;

wherein the first set of instructions cause the host system to generate a first alert, the first alert containing the allergy and the severity of the allergy but no user information;

wherein the first set of instructions cause the host system to generate a second alert, the second alert containing the allergy and the severity of the allergy as well as the user information and the treatment for the allergic reaction associated with the allergy; and

a predetermined amount of time before the at least one event, the first set of instructions cause the host system to transmit the first alert to all members of the group and the second alert only to a leader of the group.

From the above description, it is clear that the inventive concept(s) disclosed herein are well adapted to carry out the objects and to attain the advantages mentioned herein, as well as those inherent in the inventive concept(s) disclosed herein. While the embodiments of the inventive concept(s) disclosed herein have been described for purposes of this disclosure, it will be understood that numerous changes may be made and readily suggested to those skilled in the art which are accomplished within the scope and spirit of the inventive concept(s) disclosed herein.

What is claimed is:

1. A method, comprising:
establishing communication between a host system having a processor and a plurality of user devices each having an input device, an output device, and a communication device;
accepting, via the input device of a first one of the plurality of user devices associated with a first user, input comprising user information, allergy information including an allergy and a severity of the allergy, and at least one group associated with the first user;
storing, at the host system, the user information, allergy information, and the at least one group in a user profile of the first user;
automatically, without user intervention, generating an alert, the alert containing information indicative of the allergy and the severity of the allergy but none of the user information; and
automatically, without user intervention, transmitting the alert to at least one second one of the plurality of user devices associated with at least one second member of the at least one group associated with the first user.

2. The method of claim 1, wherein the alert is a first alert, the method further comprising the steps of:
generating a second alert, the second alert comprising information indicative of the allergy, the severity of the allergy, and the user information; and
transmitting the second alert to a third one of the plurality of user devices associated with a leader of the at least one group associated with the first user.

3. The method of claim 1, wherein the allergy information further includes a treatment for an allergic reaction associated with the allergy.

4. The method of claim 3, wherein the alert is a first alert, the method further comprising the steps of:
generating a second alert, the second alert comprising information indicative of the allergy, the severity of the allergy, the treatment for an allergic reaction associated with the allergy, and the user information; and
transmitting the second alert to a third one of the plurality of user devices associated with a leader of the at least one group associated with the first user.

5. The method of claim 1, further comprising the steps of:
generating and storing information indicative of a date and time of at least one event associated with the at least one group associated with the first user; and
re-transmitting, a predetermined amount of time before the at least one event, the alert containing information indicative of the allergy and the severity of the allergy, but none of the user information, to the at least one second one of the plurality of user devices associated with at least one second member of the at least one group associated with the first user.

6. The method of claim 1, further comprising the step of generating and storing information indicative of a date and time of at least one event for the at least one group associated with the first user, wherein the alert is generated and transmitted a predetermined amount time before the at least one event.

7. The method of claim 6, further comprising the step of associating at least one second user with the at least one event, wherein the alert is generated and transmitted a predetermined time before the at least one event only to the at least one second user.

8. A system for automatically distributing allergy information, comprising:
a host system having a non-transitory storage media storing information indicative of registered users associated with a group; and,
a plurality of user devices, at least one of the user devices being a first user device and at least one of the user devices being a second user device different from the first user device;
wherein the host system is programmed to receive information from the first user device over a network indicative of allergy information from a first one of the registered users and store the allergy information within a database stored on the non-transitory storage media, the allergy information comprising an allergy, an indication of a severity of a reaction to the allergy, and a treatment for an allergic reaction to the allergy; and
the host system further programmed to automatically and without user intervention disseminate an alert comprising the allergy and the indication of a severity of a reaction to the allergy over the network to at least one of the second user devices, the second user devices being associated with a defined group of the registered users associated with the group, the defined group of the registered users being second users.

9. The system for automatically distributing allergy information of claim 8, wherein the alert is a first alert and the host system is further programmed to:
generate a second alert, the second alert comprising information indicative of the allergy, the severity of the allergy, and the user information; and
transmit the second alert to a third one of the plurality of user devices associated with a leader of at least one group associated with the first user and the second user.

10. The system for automatically distributing allergy information of claim 8, wherein the allergy information further includes a treatment for an allergic reaction associated with the allergy.

11. The system for automatically distributing allergy information of claim 10, wherein the alert is a first alert and the host system is further programmed to:
generate a second alert, the second alert comprising information indicative of the allergy, the severity of the allergy, the treatment for an allergic reaction associated with the allergy, and the user information; and
transmit the second alert to a third one of the plurality of user devices associated with a leader of at least one group associated with the first user.

12. The system for automatically distributing allergy information of claim 8, wherein the alert is a first alert and the host system is further programmed to:
generating and storing information indicative of a date and time of at least one event associated with the at least one group associated with the first user; and
re-transmitting, a predetermined amount of time before the at least one event, the alert containing information indicative of the allergy and the severity of the allergy, but none of the user information, to the at least one second one of the plurality of user devices associated with at least one second member of the at least one group associated with the first user.

13. The system for automatically distributing allergy information of claim 8, wherein the host system is further programmed to generate and store information indicative of a date and time of at least one event for at least one group associated with the first user; and
wherein the host system is programmed to transmit the alert a predetermined amount time before the date and time of the at least one event.

14. The system for automatically distributing allergy information of claim 13, wherein the host system is further programmed to associate at least one second user with the at least one event, wherein the alert is generated and transmitted a predetermined time before the at least one event only to the at least one second user.

15. A system, comprising:
a host system comprising:
a first processor and at least one first non-transitory computer readable medium storing a first set of instructions that when executed by the first processor cause the first processor to obtain and record in a database event information associated with a group, the event information having parameters indicative of a date and time of at least one event that is to be attended by members of the group;
a user device comprising:
a second processor, an input device, an output device, a communication device, and at least one second non-transitory computer readable medium storing a second set of instructions that when executed by the second processor cause the second processor to obtain user information associated with a user of the user device and allergy information associated with the user, the allergy information comprising an allergy, a severity of the allergy, and a treatment for an allergic reaction associated with the allergy;
wherein the first set of instructions cause the host system to generate a first alert, the first alert containing the allergy and the severity of the allergy but no user information;
wherein the first set of instructions cause the host system to generate a second alert, the second alert containing the allergy and the severity of the allergy as well as the user information and the treatment for the allergic reaction associated with the allergy; and
a predetermined amount of time before the at least one event, the first set of instructions cause the host system to transmit the first alert to all members of the group and the second alert only to a leader of the group.
